(19) United States
(12) Patent Application Publication
(10) Pub. No.: US 2022/0241003 A1
(43) Pub. Date: Aug. 4, 2022

(54) HIGH PERMITTIVITY ELECTROSURGICAL ELECTRODE COATING

(71) Applicant: Conmed Corporation, Utica, NY (US)

(72) Inventor: Michael L. Koltz, JR., Aurora, CO (US)

(73) Assignee: Conmed Corporation, Utica, NY (US)

(21) Appl. No.: 17/622,495

(22) PCT Filed: Mar. 27, 2020
(86) PCT No.: PCT/US20/25155

§ 371 Date: Dec. 23, 2021

Related U.S. Application Data
(60) Provisional application No. 62/825,839, filed on Mar. 29, 2019.

(51) Int. Cl. A61B 18/14

(52) U.S. Cl. CPC A61B 18/14; A61B 2018/00136

(57) ABSTRACT

A coating for the electrode of an electrosurgical instrument that increases the capacitance of the electrode. The coating comprises a high permittivity material such as barium titanate, lead zirconate titanate, calcium copper titanate, or a conjugated polymer. The coating may have a thickness of 0.0016 inches and can be included with one of more insulative layers.

### CROSS-REFERENCE TO RELATED APPLICATIONS

The present application claims priority to U.S. Provisional Application No. 62/825,839, filed on Mar. 29, 2019.

### BACKGROUND OF THE INVENTION

### 1. Field of the Invention

The present invention relates to electrosurgical instruments and, more specifically, to a coating for electrosurgical electrodes having high permittivity.

### 2. Description of the Related Art

Electrosurgical instruments such as vessel sealers have become a commonly used tool for surgical procedures. These devices operate by delivering electromagnetic energy to one or more electrodes that are directly and capacitively coupled to the tissue to be treated for the purposes of performing cutting and/or coagulation of tissue to be treated through capacitive coupling. While all electrodes conduct electricity via direct (resistive) and capacitive coupling, most electrodes rely predominantly on resistive coupling which inherently generates resistive heat. Accordingly, there is a need in the art for an approach that can increase the capacitance coupling of the electrode to reduce the amount of resistive heat that is generated.

### BRIEF SUMMARY OF THE INVENTION

The present invention comprises the improvement of capacitance of an electrode of an electrosurgical instrument. The instrument has an electrode and a coating is applied to the electrode. The coating comprises a high permittivity material. The coating may have a thickness of 0.0016 inches. The coating may comprise barium titanate. The coating may comprise lead zirconate titanate. The coating may comprise a conjugated polymer. The coating may comprise lead calcium copper titanate.

The present invention also includes a method of enhancing the capacitance of an electrosurgical instrument. The method includes the step of coating an electrode of the electrosurgical instrument with a high permittivity material. The coating may have a thickness of 0.0016 inches. The coating may comprise barium titanate. The coating may comprise lead zirconate titanate. The coating may comprise a conjugated polymer. The coating may comprise lead calcium copper titanate.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a schematic of the present invention used in connection with a monopolar electrosurgical system according to the present invention;

FIG. 2 is a schematic of the present invention used in connection with a bipolar electrosurgical system according to the present invention;

FIG. 3 is a schematic of an electrode coated with a high permittivity material according to the present invention;

FIG. 4 is a schematic of an electrode coated with a high permittivity material and optional insulative layers according to the present invention.

### DETAILED DESCRIPTION OF THE INVENTION

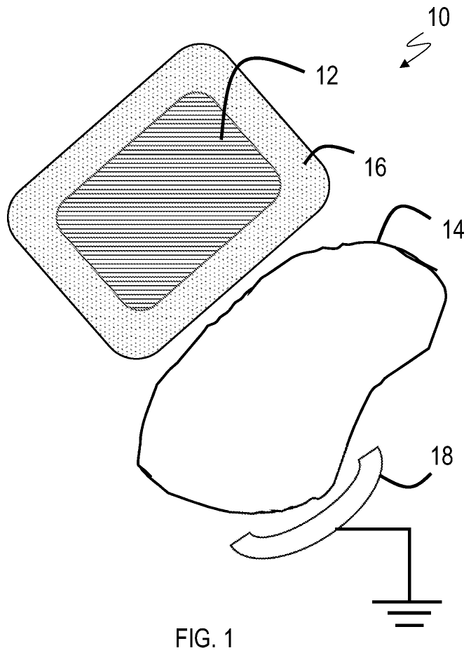

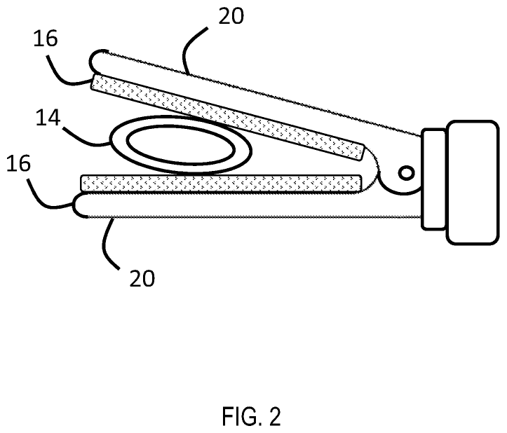

Referring to the figures, wherein like numeral refer to like parts throughout, there is seen in FIG. 1 a system 10 for improving capacitive coupling between the electrode 12 of an electrosurgical device and tissue 14 to be treated. More particularly, a high permittivity coating 16 is positioned between electrode 12 and tissue 14, such as by applying coating 16 to electrode 12 prior to use. Coating 16 may be applied to the electrode in a monopolar arrangement, as seen in FIG. 1 where a return electrode 18 is used. Coating 16 may also be used in combination with electrodes 16 of a bipolar arrangement, as seen in FIG. 2, where the jaws 20 of instrument carry electrodes 12 that are covered by coating 16 and enclose tissue 14 to be treated. Coating 16 may applied to any electrosurgical electrodes 12 functioning partially or wholly through capacitive coupling including those intended for use to cut, coagulate, or seal tissue. Coating 16 increases the capacitance of electrode 12 and provides beneficial effects, such as increasing the capacitively coupled current while reducing the direct current through the electrode, thereby resulting in lower resistive heating and a lower electrode surface temperature.

Coating 16 comprises a high permittivity material (HPM), such as ceramic or polymer, and may be applied directly to the surface of electrode 12 that will come into contact with tissue 14. Specific conjugated polymers may comprise cyano-polyphenylene vinylene, polyacetylenes, polyaniline, polyfluorenes, polyfluorene vinylene, polyfluorenylene ethynylene, polyphenylene ehynylene, polyphenylene sulfide, polyphenylene vinylene, polypyridines, polypyrroles, and polythiophenes. The relative (to free space) permittivity of the HPM is preferably at least 1000. For example, the HPM used for coating 16 may be barium titanate with a relative permittivity between 1000 and 10,000. Alternatively, the HPM used for coating 16 may be one or more of the materials listed in Table 1 below:

TABLE 1
Relative Permittivity

| Material | Nominal | Lower Limit | Upper Limit |
|---|---|---|---|
| Lead | 2500 | 500 | 6000 |
| Zirconate | | | |
| Titanate | | | |
| Barium | 5000 | 1000 | 10000 |
| Titanate | | | |
| Conjugated | 10000 | 50000 | 100000 |
| Polymer | | | |
| Calcium | 250000 | 500000 | 1000000 |
| Copper | | | |
| Titanate | | | |

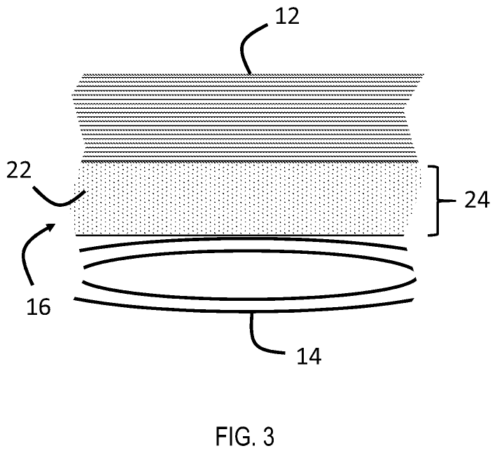

As seen in FIG. 3, coating 16 includes a plurality of suspended particles 22 within a matrix 24. Matrix 24 may comprise a silicone thermoset dispersion vulcanized at room temperature or accelerated at elevated temperature. Matrix 24 could also be molded thermoplastic, specifically a fluoropolymer such as polytetrafluoroethylene (PTFE), ethylene tetrafluoroethylene (ETFE), ethylene chlorotrifluoroethylene (ECTFE) or polyvinylidene fluoride (PVDF). Suspended particles 22 comprises 20 to 70 percent of coating 16 by volume.

The HPM material increases capacitance of electrode 16. For example, an electrode 12 having a capacitive area of 0.0455 square inches and a coating 16 of an HPM with a relative permittivity of 5000 and a thickness of 0.0016 inches with have an electrode capacitance of 812 pico-Farads. An equivalent electrode having a non-HPM, such as polytetrafluoroethylene (PTFE), will have an electrode capacitance of only 0.3 pico-Farads.

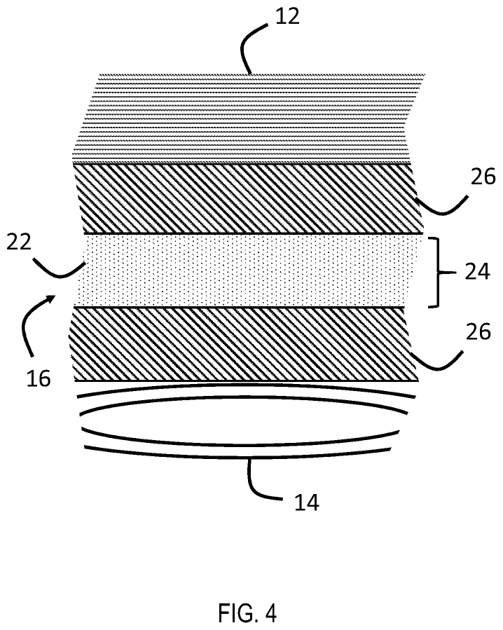

Coating 16 may also be used in combination with one or more insulative layers 26 positioned between electrode 12 and coating 16, and/or between coating 16 and tissue 14 to be treated, as seen in FIG. 4.

What is claimed is:

1. An electrosurgical instrument, comprising:
an electrode;
a coating applied to the electrode, wherein the coating comprises a high permittivity material.

2. The electrosurgical instrument of claim 1, wherein the coating has a thickness of 0.0016 inches.

3. The electrosurgical instrument of claim 1, wherein the coating comprises barium titanate.

4. The electrosurgical instrument of claim 1, wherein the coating comprises lead zirconate titanate.

5. The electrosurgical instrument of claim 1, wherein the coating comprises a conjugated polymer.

6. The electrosurgical instrument of claim 1, wherein the coating comprises lead calcium copper titanate.

7. A method of enhancing the capacitance of an electrosurgical instrument, comprising the step of coating an electrode of the electrosurgical instrument with a high permittivity material.

8. The method of claim 7, wherein the coating has a thickness of 0.0016 inches.

9. The method of claim 7, wherein the coating comprises barium titanate.

10. The method of claim 7, wherein the coating comprises lead zirconate titanate.

11. The method of claim 7, wherein the coating comprises a conjugated polymer.

12. The method of claim 7, wherein the coating comprises lead calcium copper titanate.
